(19) United States
(12) Patent Application Publication
(10) Pub. No.: US 2003/0100953 A1
(43) Pub. Date: May 29, 2003

(54) Knee joint prostheses

(76) Inventors: Richard A. Rosa, Short Hills, NJ (US); Vernon R. Hartdegen, Collierville, TN (US); Eric A. Stookey, Cordova, TN (US); Jack M. Bert, Woodbury, MN (US); Michael Gross, Halifax (CA)

Correspondence Address: Robert H. Epstein, Epstein & Gerken, Suite 340, 1901 Research Blvd., Rockville, MD, 20850, US

(21) Appl. No.: 10/305,370

(22) Filed: Nov. 27, 2002

Related U.S. Application Data
(60) Provisional application No. 60/333,487, filed on Nov. 28, 2001.

(51) Int. Cl. A61F 2/38

(52) U.S. Cl. 623/20.3; 623/20.32; 623/20.36

(57) ABSTRACT

A knee joint prosthesis comprises a femoral component for implantation on a prepared femoral condyle of a knee and a tibial component for implantation on a prepared corresponding tibial plateau of the knee. The femoral component has a femoral articular surface cooperable with a tibial articular surface of the tibial component to permit motion at the knee. The femoral articular surface has a medial-lateral radius of curvature, and the tibial articular surface has a medial-lateral radius of curvature 0.25 to 5.00 mm longer than the medial-lateral radius of curvature of the femoral articular surface. The tibial articular surface has a medial-lateral mid-point, and the femoral articular surface defines a tracking line for the femoral component which tracks in an anterior-posterior direction along the medial-lateral mid-point as the knee moves between extension and flexion. The tracking line tracks at a medial-lateral angle relative to the medial-lateral mid-point of the tibial articular surface.

### CROSS-REFERENCE TO RELATED PATENT APPLICATIONS

This application claims priority from prior provisional patent application Serial No. 60/333,487 filed Nov. 28, 2001, the entire disclosure of which is incorporated herein by reference. This application is related to the co-pending non-provisional patent applications filed concurrently herewith and entitled Instrumentation for Minimally Invasive Unicompartmental Knee Replacement (Attorney Docket No. 2333.0028C) and Methods of Minimally Invasive Unicompartmental Knee Replacement (Attorney Docket No. 2333.0029C), the entire disclosures of which are incorporated herein by reference.

### BACKGROUND OF THE INVENTION

1. Field of the Invention

The present invention relates generally to knee joint prostheses and, more particularly, to unicompartmental knee joint prostheses and to prosthetic femoral and tibial components.

2. Discussion of the Related Art

The natural knee joint includes an upper or proximal part of the tibia, constituted by the medial and lateral tibial plateaus, and a lower or distal part of the femur, constituted by the medial and lateral femoral condyles which bear upon the corresponding tibial plateaus through the intermediary of cartilage or meniscus. Connection through the knee is provided by ligaments, which also provide joint stability and assist in absorbing stresses to which the knee is subjected. The femur, tibia and cartilage are normally subjected to significant forces in supporting the weight of the body and in executing movements of the leg. The knee joint, therefore, is highly susceptible to damage from trauma and is also susceptible to damage from disease.

Knee joint prostheses for partially or totally replacing a knee joint which has been damaged due to trauma or disease have been proposed and typically include a femoral component attached to the lower part of the femur and a tibial component attached to the upper part of the tibia in articulating relation with the femoral component. The femoral components for conventional knee joint prostheses used in total knee replacement procedures may include two interconnected condylar portions for respectively bearing against upper surfaces of one or more tibial components. Knee joint prostheses of the latter type are represented by U.S. Pat. No. 6,102,955 to Mendes et al, U.S. Pat. No. 5,964,808 to Blaha et al, U.S. Pat. No. 5,567,259 to Ferrante et al, U.S. Pat. No. 5,549,687 to Coates et al, U.S. Pat. No. 5,405,395 to Coates, U.S. Pat. No. 5,370,699 to Hood et al, U.S. Pat. No. 5,219,362 to Tuke et al, U.S. Pat. No. 5,100,409 to Coates et al, U.S. Pat. No. 5,059,196 to Coates, U.S. Pat. No. 4,926,847 to Luckman, U.S. Pat. No. 4,838,891 to Branemark et al, U.S. Pat. No. 4,731,086 to Whiteside et al, U.S. Pat. No. 4,524,466 to Petersen, U.S. Pat. No. 4,340,978 to Buechel et al, U.S. Pat. No. 4,309,778 to Buechel et al, and U.S. Pat. No. 3,774,244 to Walker.

In addition to total knee replacement, unicompartmental or partial knee replacement is known wherein a single compartment of the knee is surgically restored. For example, a medial or lateral portion of the tibial-femoral joint may be replaced without sacrificing normal remaining anatomical structure in the knee. The femoral components of knee joint prostheses used in unicompartmental or partial knee replacement procedures have a single condylar portion, and single condylar femoral components may also be used in total knee replacement procedures. Knee joint prostheses having single condylar or unicondylar femoral components are represented by U.S. Pat. No. 6,059,831 to Braslow et al, U.S. Pat. No. 5,520,695 to Luckman, U.S. Pat. No. 5,395,376 to Caspari et al, U.S. Pat. No. 5,336,266 to Caspari et al, U.S. Pat. No. 5,312,411 to Steele et al, U.S. Pat. No. 5,304,181 to Caspari et al, U.S. Pat. No. 5,263,498 to Caspari et al, U.S. Pat. No. 5,234,433 to Bert et al, U.S. Pat. No. 5,228,459 to Caspari et al, U.S. Pat. No. 5,207,711 to Caspari et al, U.S. Pat. No. 5,201,768 to Caspari et al, U.S. Pat. No. 5,171,276 to Caspari et al, U.S. Pat. No. 5,171,244 to Caspari et al, U.S. Pat. No. 5,122,144 to Bert et al, U.S. Pat. No. 5,092,895 to Albrektsson et al, U.S. Pat. No. 5,037,439 to Albrektsson et al, U.S. Pat. No. 4,838,891 to Branemark et al, U.S. Pat. No. 4,743,261 to Epinette, U.S. Pat. No. 4,719,908 to Averill et al, and U.S. Pat. No. 3,958,278 to Lee et al, WO 00/30570, the Biomet Repicci II, the M/G Unicompartmental Knee of Zimmer, Inc., and the Johnson & Johnson P.F.C.

The tibial components of many knee joint prostheses are modular tibial components including a base member, typically made of metal, and an insert, typically made of plastic, mounted on the metal base member. Modular tibial components are illustrated by U.S. Pat. No. 3,958,278 to Lee et al, U.S. Pat. Nos. 4,309,778 and 4,340,978 to Buechel et al, U.S. Pat. No. 4,728,332 to Albrektsson, U.S. Pat. No. 4,743,261 to Epinette, U.S. Pat. No. 4,795,468 to Hodorek et al, U.S. Pat. No. 5,037,439 to Albrektsson et al, U.S. Pat. No. 5,047,057 to Lawes, U.S. Pat. No. 5,074,880 to Mansat, U.S. Pat. No. 5,092,895 to Albrektsson et al, U.S. Pat. Nos. 5,171,276, 5,201,768 and 5,207,711 to Caspari et al, U.S. Pat. No. 5,219,362 to Tuke et al, U.S. Pat. No. 5,336,266 to Caspari et al, U.S. Pat. No. 5,370,699 to Hood et al, U.S. Pat. No. 5,531,793 to Kelman et al, U.S. Pat. No. 5,964,808 to Blaha et al, U.S. Pat. No. 6,102,954 to Albrekstsson et al, and U.S. Pat. No. 6,102,955 to Mendes et al, the Biomet Repicci II, the M/G Unicompartmental Knee of Zimmer, inc. and the Johnson & Johnson P.F.C. The Mansat, Tuke et al and Hood et al patents, as well as the M/G Unicompartmental Knee of Zimmer, Inc., the Johnson & Johnson P.F.C., U.S. Pat. No. 3,774,244 to Walker and WO 00/30570, disclose one-piece tibial components. The Mansat, Tuke et al, Hood et al and Walker patents disclose the one-piece tibial components as being integrally formed of a synthetic plastic material. The M/G Unicompartmental Knee of Zimmer, Inc. and the Johnson & Johnson P.F.C. provide all-polyethylene tibial component options. The Mansat patent also discloses anchoring devices by which the tibial components are secured to the bone. U.S. Pat. No. 4,838,891 to Branemark et al discloses implantation of anchorage devices in the tibia and femur in a first operation followed by assembly of tibial and femoral articulating devices to the anchoring devices in a second operation.

Natural movement of an anatomically intact knee is a complex action including rolling, sliding and axial rotation. As the leg moves from full extension towards full flexion, there is pivotal rotation of the tibia about the femur, which is then converted to a rolling movement wherein the femoral condyles roll posteriorly on the tibial plateaus. The rolling movement then changes to a combined sliding and pivoting movement wherein the femoral condyles slide forward on the tibial plateaus until full flexion is obtained. Also, there may be internal/external rotation and/or varus/valgus angulation at the knee joint. This complex, polycentric motion is difficult to replicate prosthetically, and many conventional knee joint prostheses have numerous disadvantages. Some of the disadvantages associated with conventional knee joint prostheses include an imbalance of the forces transmitted from the femoral components to the tibial components, inadequate femoral-tibial tracking, the failure to maintain an adequate contact area between the articulating surfaces of the tibial and femoral components, the inability to allow a desirable range of sliding, rollback, internal/external rotation and/or varus/valgus malalignment to replicate natural anatomical motion, the lack of ligamentous stability, premature wear of articulating surfaces, anterior/distal overhang, patella articular disruption, the inability to use a universal or non-anatomical tibial component with anatomical femoral components, the need for large incisions, and the need to remove a large amount of bone to accommodate the prostheses.

In many patients, unicompartmental knee replacement is preferable to total knee replacement since more of the natural anatomical structure of the knee may be preserved. Where unicompartmental knee replacement may be accomplished with minimal bone removal, sufficient bone may remain for potential future surgical intervention, such as future total knee replacement. Unicompartmental knee replacement may be a viable interim procedure to delay the need for a total knee replacement in many patients, as it is easier to later revise a unicompartmental knee replacement to a total knee replacement than it is to revise a total knee replacement to another total knee replacement. Other advantages of unicompartmental knee replacement over total knee replacement include easier recuperations and quicker recovery times for patients, decreased hospital stays, elimination of the need for formal physical therapy in many patients after hospital discharge, retention of the cruciate ligaments, preservation of nearly normal kinematics, and use of minimally invasive incisions to access the operative site.

Unfortunately, conventional unicompartmental knee replacement techniques are very technically demanding and the instrumentation and prostheses used in conventional unicompartmental knee replacements have various drawbacks such that reproducible clinical results are difficult to attain. Unicompartmental knee replacement systems designed for minimal exposure have historically provided limited instrumentation, making reproducible alignment difficult, or bulky instrumentation, which requires more intrusive surgery. Furthermore, many prostheses used in conventional unicompartmental knee replacements require significant bone removal such that quality bone must be unduly sacrificed. Most prostheses used in conventional unicompartmental knee replacements rely on flat-on-flat or round-on-flat tibial-femoral surfaces which provide less than optimal contact area throughout a range of motion and greater polyethylene stresses. Consequently, many conventional unicompartmental prostheses tend to exhibit increased wear and decreased survivorship. A primary cause of revision in unicompartmental knee replacement has been attributed to failures due to tibial component loosening. In tibial components that utilize inlay fixation techniques, additional concerns are presented with respect to potential cancellous bone subsidence.

The Biomet Repicci II allows for a minimally invasive surgical technique but relies on a round-on-flat tibiofemoral articulation. In addition, the pegless tibial base of the Biomet Repicci II must rest completely in a pocket in the cancellous and/or sclerotic bone. The M/G Unicompartmental Knee of Zimmer, Inc. also provides a knee joint prosthesis for use in a minimally invasive surgical technique. However, some of the disadvantages of the Zimmer M/G prosthesis include less than optimal round-on-flat tibiofemoral geometry and tracking, the need for three planar resections, i.e. distal femoral condyle resection, posterior femoral condyle resection and posterior chamfer resection, in the femur to accommodate the femoral fixation surface of the femoral component, lack of anatomic femoral geometry, femoral curvature based on an older kinematic theory, significant bone removal to accommodate the femoral fixation surface as well as a pair of femoral fixation pegs on the femoral component, less than optimal tibial component fixation, and significant removal of bone from the tibia to accommodate a conical post in addition to dual tibial fixation pegs of the tibial component. The Johnson & Johnson P.F.C. includes the drawbacks of round-on-flat tibiofemoral articulation, femoral curvature based on the older kinematic theory, the need for full femoral and tibial resections with significant bone removal, and the lack of minimally invasive instrumentation.

Accordingly, the need exists for unicompartmental knee joint prostheses which provide a conservative approach in terms of bone removal and exposure while providing consistent alignment and reproducible clinical results achievable in an instrumented minimal incision technique. Particularly, the need exists for knee joint prostheses for unicompartmental knee replacement in which, among other things, bone is conserved while realizing optimal fixation of the tibial and femoral components, contact area is increased through a range of motion while allowing freedom in implant placement, essentially normal anatomy and kinematics are restored, implant wear may be reduced, and implant survivorship may be increased.

### SUMMARY OF THE INVENTION

Accordingly, it is a primary object of the present invention to overcome the above-mentioned disadvantages of prior art knee joint prostheses.

Another object of the present invention is to increase the contact area between articular surfaces in a knee joint prosthesis.

A further object of the present invention is to facilitate anatomical femoral-tibial tracking in a knee joint prosthesis.

It is also an object of the present invention to enhance stability in a knee joint prosthesis.

An additional object of the present invention is to provide ligamentous stability throughout a range of motion in a knee having an implanted knee joint prosthesis.

It is also an object of the present invention to reduce wear of a polyethylene articular surface of a tibial component of a knee joint prosthesis.

Still another object of the present invention is to articulate a femoral component along a mid-point of a tibial articular surface in a knee joint prosthesis throughout a range of motion.

The present invention has as another object to maintain maximum contact between the femoral and tibial components of a knee joint prosthesis while allowing internal/external rotation and/or varus/valgus malalignment between the components.

A still further object of the present invention is to limit or control the amount of sliding motion allowed between tibial and femoral components of a knee joint prosthesis.

Moreover, it is an object of the present invention to replicate natural sliding motion of the knee in a knee joint prosthesis.

An additional object of the present invention is to allow a universal tibial component to be used with different anatomical femoral components and/or with femoral components of different sizes.

Still another object of the present invention is to deter anterior overhang in a knee joint prosthesis.

The present invention has as a further object to deter medial-lateral overhang in a knee joint prosthesis.

Yet another object of the present invention is to avoid patella articular disruption from a knee joint prosthesis.

It is an additional object of the present invention to optimize medial kinematics in a knee joint prosthesis.

Furthermore, it is an object of the present invention to enhance alignment and cementitious fixation of a femoral component while minimizing the amount of bone that must be removed from the femur.

The present invention also has as an object to enhance cementitious fixation of a tibial component using cortical tibial rim support and dual peg fixation structure in an onlay fixation.

Yet another object of the present invention is to minimize the amount of bone which must be removed from the femur to accommodate a femoral component of a knee joint prosthesis.

Still a further object of the present invention is to minimize the amount of bone which must be removed from the tibia to accommodate a tibial component of a knee joint prosthesis while obtaining optimal fixation of the tibial component on the bone.

The present invention also has as an object to provide an optimal round-on-round tibiofemoral articular surface with increased contact area in a knee joint prosthesis.

Moreover, it is an object of the present invention to closely match the medial-lateral curvature of the tibial and femoral components of a knee joint prosthesis.

Some of the advantages of the present invention are that the femoral component may conserve about twenty percent more quality bone stock compared to conventional full resection femoral components, the minimal posterior resection required for the femoral component and the distal resurfacing geometry of the femoral component preserve bone that otherwise must be removed for conventional full resection femoral components, the femoral component essentially matches the articulating surface of the normal knee, the femoral component is a true resurfacing component with a thin profile, the femoral fixation peg and fin facilitate alignment and provide optimal fixation for the femoral component with minimal bone removal, the femoral fixation fin provides added structural integrity and strength, the femoral component has a constant sagittal radius from ten degrees to a size-dependent range of ninety to one hundred five degrees of flexion that essentially replicates the anatomical shape of the femur and restores essentially normal knee motion, the longer extension radius of the femoral component essentially replicates femoral anatomy while allowing quality bone to be preserved, the femoral component is designed to articulate along the mid-point of the tibial articular surface throughout a range of motion, the angled anterior-posterior anatomic geometry of the femoral component provides maximum implant coverage without medial-lateral overhang, the tibial component provides higher disassociation forces and optimal attributes for cement fixation, the tibial component is designed for onlay fixation with cortical tibial rim support and dual fixation pegs providing increased fixation for consistent, long-term clinical success, failures of unicompartmental knee arthroplasty due to tibial component loosening may be avoided, an all-poly tibial component conserves bone while providing proven outcomes based on cortical bone support in place of inlay fixation techniques, increased medial-lateral tibiofemoral congruency modernizes contact area to total knee implant standards, the semi-congruent anterior-posterior tibiofemoral interface provides superior contact area while allowing freedom in implant placement, anatomical tibiofemoral tracking restores essentially normal motion and maximizes implant congruency throughout flexion, the optimal round-on-round tibiofemoral surface promotes reduced overall wear and improved implant survivorship, and high contact area is maintained through a range of motion for various degrees of malalignment.

These and other objects, advantages and benefits are realized with the present invention as generally characterized in a unicompartmental knee joint prosthesis for implantation in a compartment of a knee and comprising a prosthetic femoral component for implantation on a prepared femoral condyle and a prosthetic tibial component for implantation on a prepared corresponding tibial plateau. The femoral component comprises a prosthetic body having a femoral articular surface cooperable with a tibial articular surface along the corresponding tibial plateau to permit motion at the knee. The femoral component comprises a posterior portion bisected by a first plane and a distal portion bisected by a second plane disposed at an angle to the first plane. The angle is in the range of three to fifteen degrees and, in one embodiment, the angle is seven degrees. The femoral articular surface has an anterior-posterior curvature and a medial-lateral curvature. The anterior-posterior curvature of the femoral articular surface has a constant sagittal radius of curvature from ten degrees to a range of ninety to one hundred five degrees of flexion at the knee, the range of ninety to one hundred five degrees being dependent on the size of the femoral component. The range of ninety to one hundred five degrees increases as the size of the femoral component increases. The medial-lateral curvature of the femoral articular surface is constant along the femoral articular surface. The femoral articular surface defines a tracking line for the femoral component along which the femoral component tracks along the tibial articular surface. The tracking line for the femoral component is disposed in the second plane and is thusly disposed at the angle to the first plane. The angle is in the range of three to fifteen degrees and, in one embodiment, the angle is seven degrees. The femoral component has a femoral fixation surface for cementitious fixation on the prepared femoral condyle. The femoral fixation surface comprises a planar rearward section for fixation on a planar posterior surface of the prepared femoral condyle, a curved intermediate section extending anteriorly or forwardly from the rearward section and a planar forward section extending anteriorly or forwardly from the intermediate section. The intermediate section and forward section have a configuration for fixation on a distal resurfaced area of the prepared femoral condyle. The intermediate section has a plurality of tangential radii of curvature in the sagittal plane. The planar rearward section is non-perpendicular to a central longitudinal axis of the femoral component contained in the second plane. The femoral fixation surface comprises a cavity surrounded by a border for capturing cementitious material. A femoral fixation peg and a femoral fixation fin extend outwardly from the femoral fixation surface to facilitate alignment and are conservatively designed to minimize bone removal.

The tibial component comprises a prosthetic body defining a tibial articular surface cooperable with a femoral articular surface along the corresponding femoral condyle to permit motion at the knee. The tibial articular surface has an inward medial-lateral curvature that is constant along the tibial articular surface and an inward anterior-posterior sagittal curvature. The anterior-posterior sagittal curvature comprises a central curved segment disposed between and connected with anterior and posterior curved segments, respectively. The central curved segment has a first radius of curvature and the anterior and posterior curved segments each have a second radius of curvature smaller than the first radius of curvature. The second radius of curvature for the anterior segment has a center offset anteriorly from a center of the first radius of curvature by an offset distance. The second radius of curvature for the posterior segment has a center offset posteriorly from the center of the first radius of curvature by the offset distance. The medial-lateral curvature of the tibial articular surface has a radius of curvature that is 0.25-5.0 mm longer than the medial-lateral radius of curvature of the femoral articular surface. In one preferred embodiment, the medial-lateral radius of curvature of the tibial articular surface is 1.0 mm longer than the medial-lateral radius of curvature of the femoral articular surface. The tracking line of the femoral component tracks in an anterior-posterior direction along the medial-lateral mid-point of the tibial articular surface as the knee moves between extension and flexion. The tracking line tracks at a medial-lateral angle relative to the medial-lateral mid-point of the tibial articular surface. The tibial component comprises a tibial fixation surface for cementitious fixation on a planar surface of the prepared tibial plateau. The tibial fixation surface comprises a continuously planar peripheral rim for being continuously supported on the planar surface of the prepared tibial plateau, a cavity circumscribed by the rim for receiving cementitous material, and a dovetail surrounding the cavity. The rim and dovetail enhance capture of the cementitious material. A pair of tibial fixation pegs extend outwardly from the tibial fixation surface for enhanced fixation while conserving bone. The tibial articular surface is made of a non-metallic, biocompatible weight-bearing material. In one embodiment, the tibial component is made in its entirety of a non-metallic, biocompatible weight-bearing material. In another embodiment, the tibial component comprises an insert defining the tibial articular surface and a base receiving the insert and defining the tibial fixation surface, with the insert being made of non-metallic, biocompatible weight-bearing material and the base being made of a metal material.

Other objects and advantages of the present invention will become apparent from the following description of the preferred embodiments taken in conjunction with the accompanying drawings, wherein like parts in each of the several figures are identified by the same reference characters.

### BRIEF DESCRIPTION OF THE DRAWINGS

FIG. 1 is a side perspective view of a knee joint prosthesis according to the present invention.

FIG. 2 is a side view of a femoral component of the knee joint prosthesis of FIG. 1.

FIG. 3 is a top view of the femoral component.

FIG. 4 is a front view of the femoral component.

FIG. 5 is a sectional view of the femoral component taken along line A-A of FIG. 3.

FIG. 6 is a sectional view of the femoral component taken along line B-B of FIG. 2.

FIG. 7 is a sectional view of the femoral component taken along line C-C of FIG. 2.

FIG. 8 is a side view of a tibial component of the knee joint prosthesis of FIG. 1.

FIG. 9 is a top view of the tibial component.

FIG. 10 is a front view of the tibial component.

FIG. 11 is a bottom view of the tibial component.

FIG. 12 is an enlarged sectional view of the tibial component taken along line D-D of FIG. 9.

FIG. 13 is an enlarged sectional view of the tibial component taken along line E-E of FIG. 9.

FIG. 14 is an anterior view of the knee joint prosthesis implanted on a knee, with the knee being shown in extension.

FIG. 15 is an anterior view of the knee joint prosthesis implanted on the knee, with the knee being shown in flexion.

FIG. 16 is a side view of the knee joint prosthesis implanted on the knee and illustrating a range of motion in flexion.

FIG. 17 is a side view of the knee joint prosthesis implanted on the knee and illustrating a range of motion in hyperextension.

FIG. 18 is a top view of the knee joint prosthesis depicting internal/external rotation.

FIG. 19 is a front view of the knee joint prosthesis depicting varus/valgus rotation.

FIG. 20 is a sectional view of a modified tibial component for an alternative knee joint prosthesis according to the present invention taken in a coronal plane.

FIG. 21 is a sectional view of the tibial component of FIG. 20 taken in a sagittal plane.

FIG. 22 is a top view of an insert of the tibial component of FIG. 20.

FIG. 23 is a top view of a base of the tibial component of FIG. 20.

FIG. 24 is a bottom view of the base.

FIG. 25 is a sectional view of the base taken along line F-F of FIG. 23.

FIG. 26 is sectional view of the base taken along line G-G of FIG. 24.

FIG. 27 is a sectional view of another modified tibial component for a further alternative knee joint prosthesis according to the present invention taken in a coronal plane.

### DETAILED DESCRIPTION OF THE PREFERRED EMBODIMENTS

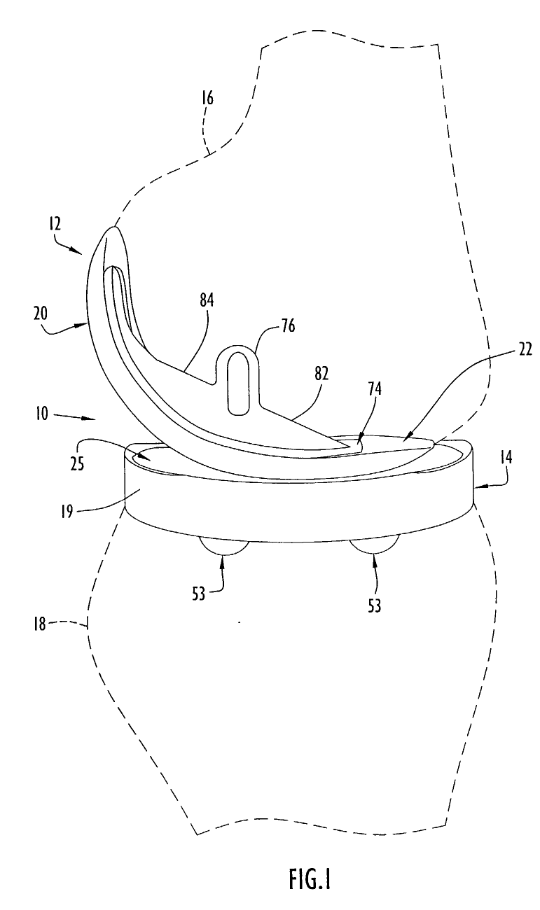

A knee joint prosthesis 10 according to the present invention is illustrated in FIG. 1 and includes a prosthetic femoral component 12 and a prosthetic tibial component 14 for implant on a femur 16 and tibia 18, respectively, of a knee. The femoral component 12 is affixed to a suitably prepared site on a condyle of the femur 16 as shown in dotted lines. The tibial component 14 is affixed to a suitably prepared site on a tibial plateau of the tibia 18 as also shown in dotted lines. The femoral component 12, as illustrated herein in FIGS. 1-7, is a “left” femoral component anatomically designed for implantation on the left knee of a patient, and a “right” femoral component (not shown) a natomically designed for implantation on the right knee of a patient is a mirror image of femoral component 12. The tibial component 14 is a universal or non-anatomical tibial component in that the same tibial component 14 may be used with anatomical “left” and “right” femoral components and/or with femoral components of different sizes as explained further below.

The knee joint prostheses of the present invention are designed to more closely replicate the geometry and kinematics of a single compartment of the knee and are thusly intended for single compartment or unicompartmental joint disease or trauma where the associated ligaments are essentially intact and functional. The tibial and femoral components of the knee joint prostheses of the present invention are illustrated herein for implantation, respectively, on the medial tibial plateau and medial femoral condyle of the left or right knee of a patient in medial compartment unicompartmental knee replacement or arthroplasty. However, the tibial and femoral components can be adapted for implantation, respectively, on the lateral tibial plateau and lateral femoral condyle of the left or right knee of a patient in lateral compartment knee replacement.

The “medial compartment” of a knee refers to the femoral condyle and corresponding tibial plateau located and corresponding tibial plateau located closer to the median plane of the patient's body, i.e. the plane that divides the body in half lengthwise, and “lateral compartment” of a knee refers to the femoral condyle and corresponding tibial plateau located further from the median plane. The term “medial” refers to a side or direction toward the median plane and the term “lateral” refers to a side or direction away from the median plane. The term “anterior” refers to a side or direction toward the front of the knee, and the term “posterior” refers to a side or direction toward the back of the knee. The term “distal” refers to a downward side or direction, and the term “proximal” refers to an upward side or direction.

The femoral component 12, as illustrated in FIGS. 1-7, is a unicondylar or single condylar femoral component comprising a single condylar prosthetic portion or prosthetic body having an outer surface 20 including a femoral articular surface and an inner surface 22 forming a femoral fixation surface. The femoral articular surface is geometrically coupled with a tibial articular surface a long the corresponding tibial plateau, and the femoral fixation surface is affixed to the prepared site on the femur 16 as explained further below. The outer surface 20 is curved in an anterior-posterior direction from a curved anterior edge 26 to a curved posterior edge 28 of femoral component 12 as best shown in FIGS. 2 and 5. Outer surface 20 includes an anterior transition segment 30, anterior and posterior articular segments 32 and 34, respectively, and a posterior transition segment 36. Anterior articular segment 32 is joined to anterior edge 26 by anterior transition segment 30, from which anterior articular segment 32 extends posteriorly. Posterior articular segment 34 extends posteriorly from anterior articular segment 32 to posterior transition segment 36. Posterior transition segment 36 extends posteriorly from posterior articular segment 34 to posterior edge 28. The anterior and posterior articular segments 32 and 34 define the femoral articular surface, and the anterior and posterior transition segments 30 and 36 provide a smooth, gentle transition between the femoral articular surface and the bone surface of the femur.

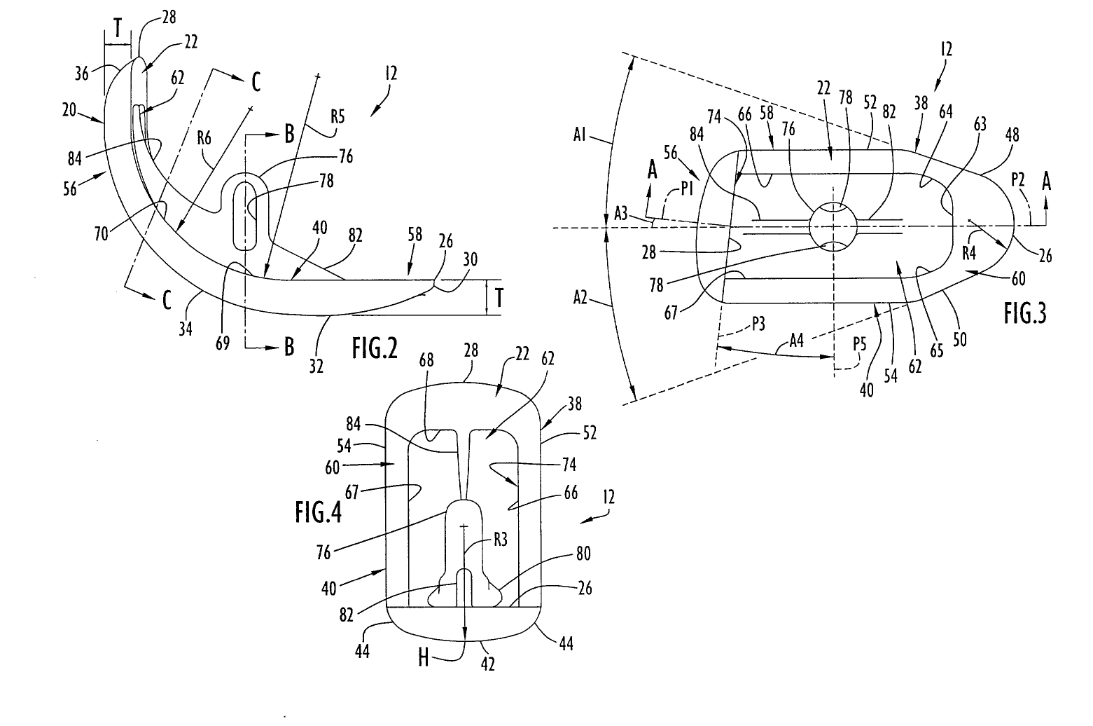

Intersecting planes P1 and P2, shown in FIG. 3, bisect femoral component 12 along a line A-A to define a sagittal profile, shown in FIG. 5, and the planes P1 and P2 may be considered sagittal planes. The anterior-posterior curvature of the femoral articular surface in the sagittal profile or plane corresponds to the curvatures of the anterior and posterior articular segments 32 and 34. The curvatures of the anterior and posterior articular segments 32 and 34 in the sagittal profile have radii of curvatures R1 and R2, respectively, with radius of curvature R2 being smaller than radius of curvature R1. Radii of curvatures R1 and R2 may be considered tangent radii of curvatures in that anterior articular segment 32 is joined to posterior articular segment 34 at a point tangent to radii of curvatures R1 and R2. As explained further below, the anterior-posterior curvature of the femoral component has a radius of curvature in the sagittal plane that is constant from ten degrees to a size-dependent range of ninety to one hundred five degrees of flexion in the implanted knee. The femoral articular surface is also curved in a medial-lateral direction between side edges 38 and 40 of femoral component 12 as best shown in FIGS. 4, 6 and 7, and the medial-lateral curvature of the femoral articular surface corresponds to the medial-lateral curvature of the femoral component. The medial-lateral curvature of the femoral component is constant throughout the femoral articular surface and is defined by a curved middle segment 42 located between two shorter, curved side segments 44 joined to the fixation surface at side edges 38 and 40 as shown in FIG. 4. The middle segment 42 has a radius of curvature R3 greater than the radius of curvature for side segments 44. A point H of the middle segment 42 centered between side edges 38 and 40 may be considered as defining the highest point of the femoral articular surface.

The anterior edge 26 is curved or rounded as shown in FIG. 3, and the curvature of anterior edge 26 has a radius of curvature R4 with a center offset from plane P2. Side edges 38 and 40 include anterior side edge segments 48 and 50, respectively, and posterior side edge segments 52 and 54, respectively. The anterior side edge segments 48 and 50 extend angularly outwardly in the posterior direction from opposite ends, respectively, of the anterior edge 26. The posterior side edge segments 52 and 54 extend posteriorly from anterior side edge segments 48 and 50, respectively, to opposite ends, respectively, of posterior edge 28. Anterior side edge segment 48 is located on the same side of plane P2 as the center for radius of curvature R4 and is disposed in a plane which defines angle A1 with plane P2. Anterior side edge segment 50 is located on the opposite side of plane P2 and is disposed in a plane which defines an angle A2, greater than angle A1, with plane P2. The posterior side edge segments 52 and 54 are parallel to one another as shown in FIGS. 3 and 4. The femoral component 12 has an overall width or medial-lateral dimension in plane P5 between side edges 38 and 40. In particular, the overall width or medial-lateral dimension of femoral component 12 corresponds to the uniform or constant width between posterior side edge segments 52 and 54, and the width of the femoral component tapers between the anterior side edge segments 48 and 50. The femoral component 12 has an overall length in plane P2 defined by the distance between anterior edge 26 and the most posterior point on the outer surface 20. A thickness T of the femoral component 12 is shown in FIG. 2.

A posterior or rearward portion 56 of femoral component 12 is angled or skewed relative to a central longitudinal axis of a distal or anterior portion 58 of femoral component 12. As seen in FIG. 3, the distal portion 58 is bisected by plane P2, which contains a central longitudinal axis of the femoral component and its distal portion, while the posterior portion 56 is bisected by plane P1, which defines an angle A3 with plane P2. The highest point H of the femoral articular surface is contained in plane P2 such that point H is disposed at angle A3 to plane P1. Angle A3 is in the range of 3 to 15 degrees and, in one preferred embodiment, angle A3 is 7 degrees. Accordingly, the highest point of the femoral articular surface is angled from 3 to 15 degrees, and is angled 7 degrees in one preferred embodiment, with respect to the posterior angle of femoral component 12 and is also centered within the width or medial-lateral dimension of the femoral component. The femoral articular surface has an anatomic geometry and, when the femoral component 12 is placed on the prepared femoral condyle, the femoral component closely replicates the natural anatomy of an intact femoral condyle.

The femoral fixation surface includes a peripheral border 60 having an outer periphery defined by anterior edge 26, posterior edge 28 and side edges 38 and 40, and an inner periphery circumscribing a recessed surface 62. As best shown in FIGS. 3 and 4, the inner periphery is defined by an inner anterior edge 63, inner anterior side edges 64 and 65, inner posterior side edges 66 and 67, and inner posterior edge 68. The inner posterior side edges 66 and 67 are parallel and are spaced a uniform or constant distance inwardly of the posterior side edge segments 52 and 54, respectively, along which the inner posterior side edges 66 and 67 respectively extend. The inner anterior side edges 64 and 65 are curved, and connect the inner posterior side edges 66 and 67, respectively, to opposite ends of inner anterior edge 63. Opposite ends of inner posterior edge 68 are curved to define rounded corners joined to inner posterior side edges 66 and 67, respectively.

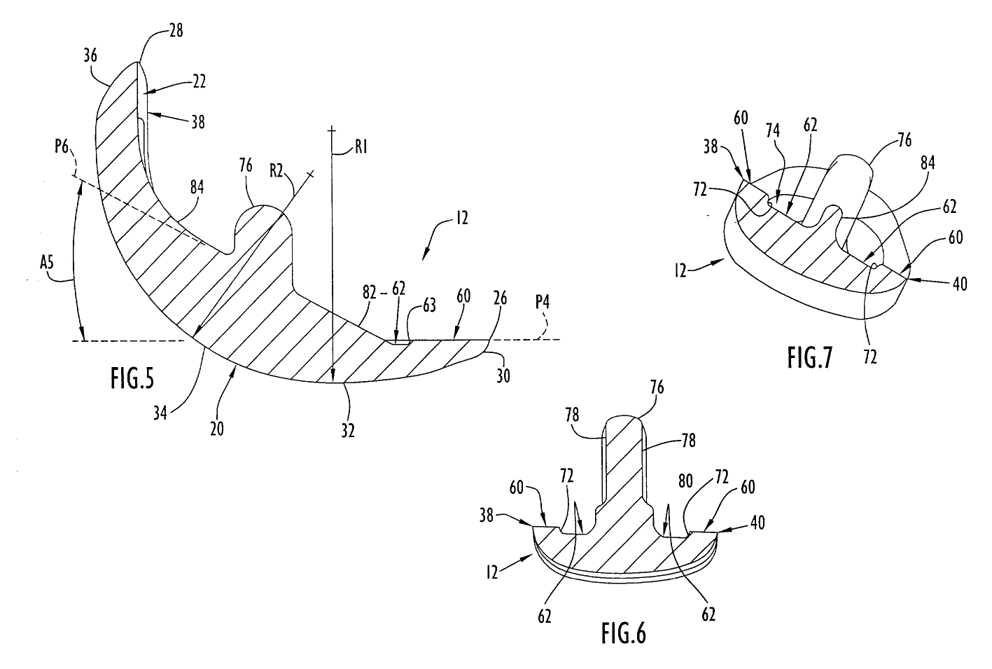

As shown in FIGS. 2 and 5, an anterior or forward section of peripheral border 60 is flat or planar extending posteriorly or rearwardly from anterior edge 26 to approximately the posterior ends of the anterior side edge segments 48 and 50. A curved intermediate section of the peripheral border 60 includes curved intermediate section segments disposed on opposite sides of recessed surface 62 as best shown in FIG. 2. The curved intermediate section segments extend posteriorly or rearwardly from approximately the posterior ends of the side edge segments 48 and 50 until the peripheral border 60 becomes flat or planar again at a posterior or rearward section of peripheral border 60, the rearward section continuing to posterior edge 28. The planar rearward section of peripheral border 60 is disposed in a plane P3 as seen in FIG. 3. As shown in FIG. 5, the planar forward section of peripheral border 60 is disposed in a plane P4. The central longitudinal axis of the femoral component is non-perpendicular to the plane P3 and is angularly offset in the medial-lateral direction from plane P3 and from a plane, i.e. plane P1, perpendicular to the plane P3 of the rearward section of border 60. The curved intermediate section segments of peripheral border 60 each include curved anterior and posterior surface segments 69 and 70 as shown in FIG. 2. Anterior surface segments 69 extend posteriorly or rearwardly from opposite ends of the planar forward section of peripheral border 60, and posterior surface segments 70 extend posteriorly or rearwardly from anterior surface segments 69, respectively, as shown in FIG. 2 for the anterior surface segment 69 and the posterior surface segment 70 along side edge 40. The anterior surface segments 69 have a radius of curvature R5, and the posterior surface segments 70 have a radius of curvature R6 smaller than and tangential to radius of curvature R5. Accordingly, the curved intermediate section of border 60 has a plurality of tangent radii of curvature in the sagittal plane. The recessed surface 62 follows the geometric configuration of peripheral border 60 and is joined to the inner periphery of peripheral border 60 by a connecting wall 72 of the femoral fixation surface as shown in FIGS. 6 and 7. A pocket or cavity 74 for receiving cement or other bonding or cementitious material used in securing the femoral component 12 on the femur 16 is defined between border 60 and surface 62 and is circumscribed by connecting wall 72.

The femoral fixation surface is adapted to mate with a medial or lateral femoral condyle prepared as disclosed in the co-pending non-provisional patent applications previously incorporated herein by reference. The planar rearward section of border 60 has a configuration corresponding to a planar posterior resected surface or area prepared along a posterior aspect of the femoral condyle while the planar forward section and curved intermediate section of border 60 have a configuration corresponding to a resurfaced area prepared along a distal aspect of the femoral condyle. As further explained in the co-pending applications, the resurfaced area is curved in the anterior-posterior direction and is formed by controllably removing a minimal surface layer of cartilage and/or bone from the distal aspect of the femoral condyle to a controlled depth while essentially retaining the anatomic femoral geometry as opposed to substantially altering the anatomic femoral geometry by resecting the distal aspect of the femoral condyle in one or more planar cuts.

A femoral fixation peg 76 extends proximally, outwardly or upwardly from the femoral fixation surface and has a central longitudinal axis disposed in plane P2 and in plane P5 perpendicular to plane P2 as shown in FIG. 3. Plane P5 is disposed at an angle A4 with plane P3, and angle A4 is the same as the angle A3. Opposing sides of femoral fixation peg 76 have external recesses 78 therein of partial circular configuration in cross-section, the recesses 78 extending distally from near the top of the fixation peg to an enlarged or thicker base 80 of the fixation peg joined to recessed surface 62. Accordingly, the recesses extend from the base to an outer end of the femoral fixation peg. A raised femoral fixation fin extends along the femoral fixation surface transverse to fixation peg 76 and includes a longitudinal anterior femoral fixation fin segment 82 and a longitudinal posterior femoral fixation fin segment 84 extending outwardly, proximally or upwardly from recessed surface 62. The anterior femoral fixation fin segment 82 extends anteriorly from fixation peg 76 to terminate on recessed surface 62 a short distance posteriorly of inner edge 63. The posterior femoral fixation fin segment 84 extends posteriorly from fixation peg 76 to the center of inner posterior edge 68. The fixation fin segments 82 and 84 are centered within the medial-lateral width of the femoral component and are thusly bisected by plane P2. As shown in FIG. 5, the terminal proximal, upper or outer edge of anterior femoral fixation fin segment 82 is contained in a plane P6 disposed at an angle A5 with plane P4. The femoral fixation peg and fin are preferably formed integrally, unitarily or monolithically with the femoral component 12 so that the femoral component is preferably a single monolithic piece.

In an illustrative but not limiting embodiment for one size of femoral component 12, angle A1 is about 19 degrees, angle A2 is about 22 degrees, angles A3 and A4 are about 7 degrees, angle A5 is about 27 degrees, radius of curvature R1 is about 1.506 inches, radii of curvatures R2 and R3 are about 0.984 inch, radius of curvature R4 is about 0.262 inch, radius of curvature R5 is about 1.427 inches, radius of curvature R6 is about 0.807 inch, and the radius of curvature for side segments 44 is about 0.172 inch. In the illustrative but not limiting embodiment, the femoral component has an overall medial-lateral width of about 0.784 inch, an overall anterior-posterior length of about 1.705 inches, and a thickness T of about 0.177 inch. Also, in the illustrative but not limiting embodiment, the center for radius of curvature R4 is offset about 0.020 inch from plane P2, the longitudinal axis of fixation peg 76 is contained in a plane parallel to plane P3, the perpendicular distance between this plane and plane P3 is about 0.545 inch and defines a femoral fixation peg location for the femoral component, the perpendicular distance between plane P5 and the inner anterior edge 63 is about 0.649 inch, the perpendicular distance between the top of fixation peg 76 and plane P4 is about 0.550 inch and defines a femoral fixation peg height for the femoral component, the perpendicular distance between plane P4 and a plane containing the inner posterior edge 68 is about 0.915 inch, the recessed surface 62 is recessed about 0.025 inch from the peripheral border 60, the inner posterior side edges 66 and 67 are located about 0.115 inch inwardly of the posterior side edge segments 52 and 54, respectively, the femoral fixation fin has a width or thickness of about 0.075 inch, the base 80 of fixation peg 76 has a diametric dimension of about 0.250 inch, and the recesses 78 have a radius of curvature of about 0.125 inch. The femoral component is typically made of metal and, in the illustrative but not limiting embodiment, the femoral component is made of ASTM F75 material.

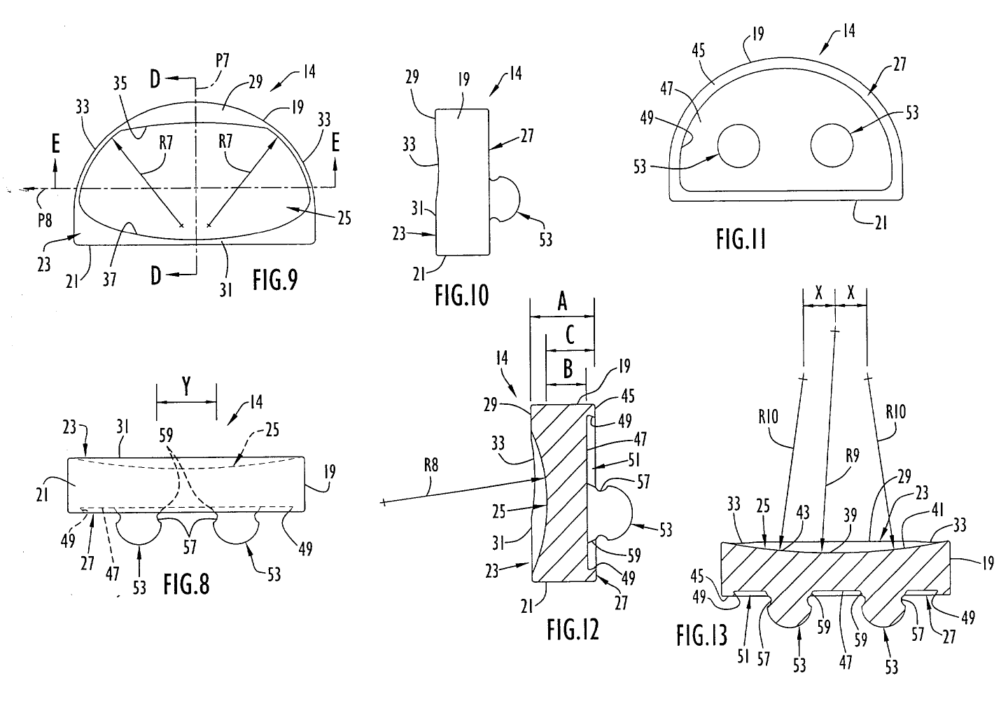

Tibial component 14, which is best illustrated in FIGS. 1 and 9-13, is a one-piece tibial component including an integral, unitary or monolithic part made in its entirety of a non-metallic, biocompatible weight-bearing material such as polyethylene material, an example of which is MS 202.10. The tibial component 14 has a D-shaped peripheral configuration when viewed from above, the D-shaped peripheral configuration being symmetrical about coronal plane P7 and about a plane P8 perpendicular to coronal plane P7 as shown in FIG. 9. The peripheral configuration of tibial component 14 is defined by an arcuate side wall 19, which is a medial side wall where the tibial component is implanted on a prepared medial tibial plateau, connected to a planar side wall 21, which is a lateral side wall where the tibial component is implanted on a prepared medial tibial plateau. The tibial component has an upper or proximal surface 23 including tibial articular surface 25 and a lower or distal surface 27 forming a tibial fixation surface for being affixed to the prepared site on the tibial plateau. The tibial articular surface 25 is bounded by a first planar segment 29 of upper surface 23 and by a co-planar second planar segment 31 of upper surface 23. Where the tibial component is implanted in the medial compartment of the knee, the tibial articular surface 25 is bounded medially by the segment 29, which is a planar medial segment of upper surface 23, and is bounded laterally by the segment 31, which is a co-planar lateral segment of upper surface 23. The segment 29 and the segment 31 are each symmetrical about coronal plane P7 and each has anterior and posterior ends merging with top edge segments 33 of arcuate side wall 19. As best seen in FIGS. 10, 12 and 13, the top edge segments 33 curve inwardly or downwardly from the segments 29 and 31. As seen in FIG. 9, each top edge segment 33 is also curved between the corresponding ends of the segments 29 and 31 with radii of curvatures R7. The radii of curvatures R7 for top edge segments 33 have centers, respectively, disposed on anterior and posterior sides, respectively, of plane P7. The tibial articular surface 25 is circumscribed by the segment 29, the segment 31 and the top edge segments 33. The tibial articular surface 25 curves inwardly or downwardly from the top edge segments 33, from a curved first edge 35 connecting the tibial articular surface 25 to the first segment 29, and from a curved second edge 37 connecting the tibial articular surface 25 to the second segment 31.

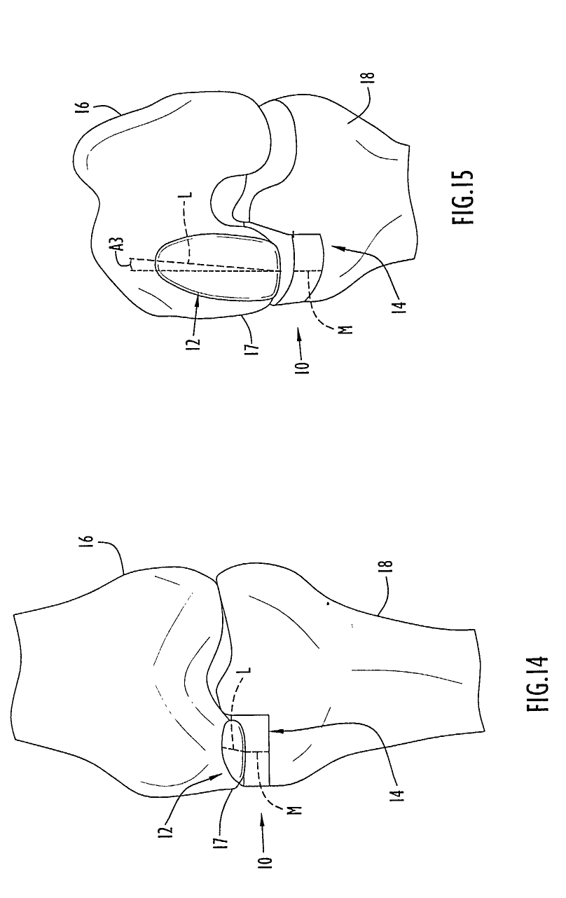

The geometric configuration of the tibial articular surface 25 is best shown in FIGS. 12 and 13. FIG. 12 depicts the medial-lateral curvature of the tibial articular surface 25, which is constant along the tibial articular surface and has a medial-lateral radius of curvature R8. Radius of curvature R8 differs from the medial-lateral radius of curvature R3 for femoral component 12 by being 0.25-5.0 mm longer than the radius of curvature R3, with the radius of curvature R8 being 1 mm longer than the radius of curvature R3 in one illustrative preferred embodiment. Accordingly, there is a mismatch or difference of 0.25-5.0 mm between the constant medial-lateral radii of curvature for the tibial and femoral articular surfaces, respectively, and in the illustrated preferred embodiment this mismatch is 1 mm.

The anterior-posterior or sagittal curvature of the tibial articular surface 25 is illustrated in FIG. 13 and includes a central curved segment 39, having radius of curvature R9, disposed between and connected with anterior and posterior curved segments 41 and 43, respectively, having radii of curvatures R10. The radii of curvatures R10 are smaller than radius of curvature R9, and the center for each radius of curvature R10 is offset from the center for radius of curvature R9 by an offset distance X. Accordingly, the center for radius of curvature R10 for anterior segment 41 is offset distance X from the center of radius of curvature R9 in an anterior direction, and the center for radius of curvature R10 for posterior segment 43 is offset distance X in a posterior direction from the center of radius of curvature R9. The distance 2X between the centers of radii of curvatures R10 controls the amount of sliding or anterior-posterior travel permitted in the knee joint prosthesis.

The lower or tibial fixation surface 27 of tibial component 14 includes a continuously planar peripheral rim 45 following the D-shaped peripheral configuration of the tibial component, a planar surface 47 recessed relative to rim 45, and a connecting wall 49 connecting recessed surface 47 to rim 45. A pocket or cavity 51 is defined between rim 45 and surface 47 and is circumscribed by connecting wall 49 to receive cement or other cementitious or bonding material used in securing the tibial component on the prepared surface of the tibial plateau. Connecting wall 49 is angled inwardly from surface 47 to form a dovetail for enhanced capture of cementitious material and fixation of the tibial component to the bone surface of the tibia. A pair of tibial fixation pegs 53 extend distally, outwardly or downwardly from recessed surface 47 and protrude distally, outwardly or downwardly relative to rim 45. Tibial fixation pegs 53 each have a rounded distal, outer or lower end connected by a narrower neck or groove 57 to an enlarged base 59 joining the fixation peg to the surface 47. The tibial fixation pegs for tibial component 14 are formed integrally, unitarily or monolithically with the tibial component. A pair of tibial fixation pegs 53 comprising a posterior tibial fixation peg and an anterior tibial fixation peg has been found to be most advantageous for enhanced fixation while conserving bone. The planar rim 45 is adapted to mate with or to be continuously supported upon a planar surface or area prepared along the prepared tibial plateau.

In an illustrative but not limiting embodiment for one size of tibial component 14, radius of curvature R8 is about 1.024 inches, radius of curvature R9 is about 5.47 inches, radii of curvatures R10 are about 2.392 inches, the centers for the radii of curvatures RIO are offset an offset distance X of about 0.197 inch from the center of radius of curvature R9, the center of radius of curvature R8 is located a perpendicular distance of about 0.925 inch from the plane of segment 29, and the top edge segments 33 have radii of curvatures R7 of about 0.827 inch with centers offset about 0.0395 inch from plane P7. The tibial component for the illustrative but not limiting embodiment has an overall anterior-posterior length of about 1.73 inches and an overall medial-lateral width of about 1.024 inches. The illustrative but not limiting tibial component has an overall thickness, dimension A in FIG. 12, between the plane of segment 29 and the plane of rim 45 of about 0.424 inch, a partial thickness, dimension C in FIG. 12, between the plane of rim 45 and the plane tangent to the medial-lateral curvature of tibial articular surface 25 of about 0.326 inch, and an internal thickness, dimension B in FIG. 12, between the plane of surface 47 and the plane tangent to the medial-lateral curvature of the tibial articular surface of about 0.281 inch. Also in the illustrative but not limiting embodiment, the connecting wall 49 is disposed at about a 45 degree angle to the plane of surface 47, the plane of rim 45 is disposed about 0.045 inch below surface 47, the rim 45 has a uniform or substantially uniform width of about 0.074 inch, the fixation pegs 53 have a maximum diametric dimension of about 0.310 inch, the necks 57 have a radius of curvature of about 0.031 inch and a minimum diametric dimension of about 0.250 inch, the pegs 53 protrude a distance of about 0.230 inch below the plane of rim 45, the centers of fixation pegs 53 are located about 0.394 inch from the planar side 21 and about 0.350 inch anteriorly and posteriorly, respectively, from plane P7. Furthermore, the tibial component of the illustrative but not limiting embodiment is characterized by an anterior-posterior range of travel, indicated by arrow Y in FIG. 8, of about 0.3937 inch.

The illustrative embodiments described above for femoral component 12 and tibial component 14 are representative of Size 2 femoral and tibial components. However, the femoral and tibial components can each be made available in different sizes. For example, in addition to Size 2, the femoral component can be made available in Size 1, Size 3 and Size 4. Femoral components in Sizes 1, 3, and 4 will differ from femoral component 12 primarily in overall medial-lateral width, overall anterior-posterior length, sagittal radius, i.e, radius of curvature R2, and/or peg height. The location of the femoral fixation peg will vary for different size femoral components.

The Size 2 tibial component described herein by way of example may be made available in different thicknesses, including 7 mm, 8 mm and 9 mm thicknesses, for example. The Size 2 tibial components of different thicknesses will differ from one another primarily in their overall thickness (dimension A), their internal thickness (dimension B) and/or their partial thickness (dimension C), the tibial component 14 being described above as an 8 mm thickness tibial component. In addition to the Size 2 tibial components, the tibial components can be made available in Sizes 1, 3 and 4, with each size made available in different thicknesses. Tibial components in Sizes 1, 3 and 4 will differ from tibial component 14 primarily in overall medial-lateral width, overall anterior-posterior length and/or anterior-posterior travel, with the medial-lateral radius, i.e. R9, being the same for each size tibial component.

Size selection for the femoral and tibial components is typically made initially based on pre-operative examinations and studies including radiograph analysis, and is finalized by the surgeon with the use of trial components during the knee replacement procedure. A selected size tibial component can be used with anatomical “left” or “right” femoral components merely by reversing the orientation of the tibial component. In addition, tibial components of different sizes and/or thicknesses can be used with a selected femoral component.

The knee joint prosthesis is preferably implanted in a medial or lateral compartment of a patient's knee in a minimally invasive procedure using the instruments and methods described in the above-referenced co-pending patent applications incorporated herein by reference. The knee joint prosthesis is implanted in the knee with minimal removal of bone to prepare the femoral condyle and tibial plateau to accommodate the femoral and tibial components, respectively. The amount of bone that must be removed to prepare the femoral condyle to mate with the border of the femoral fixation surface is significantly minimized using a minimal planar posterior resection and a distal resurfacing geometry which preserves bone ordinarily removed for traditional full resection femoral components. The femoral component allows about twenty percent more quality bone stock to be conserved as compared to conventional full resection femoral components. The femoral fixation peg and fin are conservatively designed to provide optimal alignment and cemented fixation of the femoral component on the prepared femoral condyle with minimal bone removal. The conservative design of the tibial fixation peg also minimizes bone removal. The tibial component promotes enhanced cemented fixation of the tibial component on the prepared tibial plateau and longer survivorship utilizing a proximal tibial resection, cortical tibial rim support and dual tibial fixation pegs in an onlay fixation technique which addresses the concerns normally associated with inlay techniques regarding potential cancellous and sclerotic bone subsidence. The dovetail of the tibial component enhances confinement of cementitious material in the cavity of the tibial fixation surface without penetrating the bone.

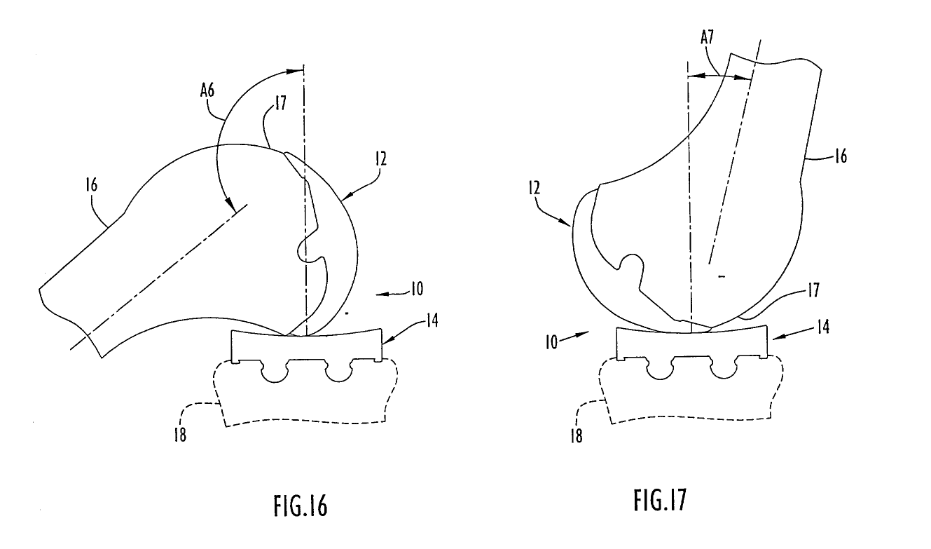

FIGS. 14 and 15 illustrate the knee joint prosthesis 10 with the femoral component 12 implanted on a prepared medial femoral condyle 17 of femur 16 and the tibial component 14 implanted on the corresponding prepared medial tibial plateau of tibia 18 of the left knee of a patient. FIGS. 14 and 15 depict a range of motion for the knee between flexion and extension, FIG. 14 showing the knee in extension and FIG. 15 showing the knee in full flexion. Actual range of motion is dictated not only by the design of the knee joint prosthesis, but also by the anatomy of the intact portion of the knee. Accordingly, the range of motion angles set forth below in the discussion of FIGS. 16-19 should be considered illustrative and not limiting.

Tibiofemoral tracking obtained with the knee joint prosthesis 10 is represented in FIGS. 14 and 15, and this tracking essentially replicates anatomical tibiofemoral tracking. As the knee moves between extension and flexion, the femoral component 12 articulates along the medial-lateral mid-point M of the tibial component 14, shown in dotted lines on the tibial component 14, throughout the range of motion, with the highest point of the femoral articular surface defining a tracking line L shown on the femoral component 12 in dotted lines. The tracking line L tracks along the medial-lateral mid-point M in an anterior-posterior direction as the knee moves between extension and flexion. The tracking line L, which follows the central longitudinal axis of the femoral component, tracks at a medial-lateral angle A3 as the knee moves between extension and flexion. Where the knee joint prosthesis is implanted in the medial compartment as shown, the tracking line L tracks laterally. Anatomical tibiofemoral tracking restores essentially normal motion and maximizes medial-lateral implant congruency throughout flexion. A close match between the medial-lateral radii of the femoral and tibial articular surfaces results in increased tibiofemoral contact area and an optimal round-on-round tibiofemoral surface that promotes reduced overall wear and improved prosthesis survivorship. FIGS. 14 and 15 also depict the anterior-posterior angle of the femoral component providing maximum implant coverage without medial-lateral implant overhang. The semi-congruent anterior-posterior tibiofemoral interface provides superior contact area between the femoral and tibial components while permitting freedom for femoral and tibial component placement. The articular surface of the femoral component has a constant sagittal radius from ten degrees to a range of ninety to one hundred five degrees of flexion at the knee in combination with the angled anterior-posterior geometry to essentially replicate the anatomical shape of the femur and restore normal motion. The ninety to one hundred five degree range in flexion is dependent on the size of the femoral component in that the range increases as the size of the femoral component increases. The longer extension radius provided anteriorly by the anterior articular segment and anterior transition segment replicates anatomy while preserving quality bone normally removed by full resection femoral components. As explained below, the knee joint prosthesis provides enhanced contact area between the femoral and tibial components throughout a range of motion despite various degrees of malalignment, as compared to prostheses in which the femoral and tibial components have less conforming medial-lateral surfaces.

FIGS. 16 and 17 are representative of the range of motion for the knee implanted with knee joint prosthesis 10 in flexion and hyperextension, respectively. As illustrated in FIG. 16, the femur 16 is rotated in flexion until just prior to contact between the tibial articular surface and the posterior femoral condyle. The range of motion of knee joint prosthesis 10 in flexion corresponds to angle A6, which is about one hundred twenty eight degrees in the illustrative but not limiting embodiment. The range of motion for knee joint prosthesis 10 in hyperextension corresponds to angle A7 in FIG. 17, angle A7 being about sixteen degrees for the illustrative but not limiting embodiment. Accordingly, the range of motion for the knee joint prosthesis 10 of the illustrative but not limiting embodiment ranges from about sixteen degrees of hyperextension to about one hundred twenty degrees of flexion.

FIG. 18 is representative of internal/external rotation of the knee implanted with knee joint prosthesis 10. FIG. 18 shows femoral component 12 rotated internally until the femoral articular surface begins to interfere with the tibial articular surface of tibial component 14. In this position, the longitudinal axis of the femoral component 12 is displaced an angle A8 of about thirteen degrees for the knee joint prosthesis of the illustrative but not limiting embodiment. Therefore, the femoral component 12 for the illustrative but not limiting embodiment internally rotates about thirteen degrees before interfering with the tibial articular surface. The femoral component 12 of the illustrative but not limiting embodiment can also rotate externally the same distance, i.e., about thirteen degrees, such that the total range of internal/external rotation for the illustrative but not limiting knee joint prosthesis 10 is about twenty six degrees.

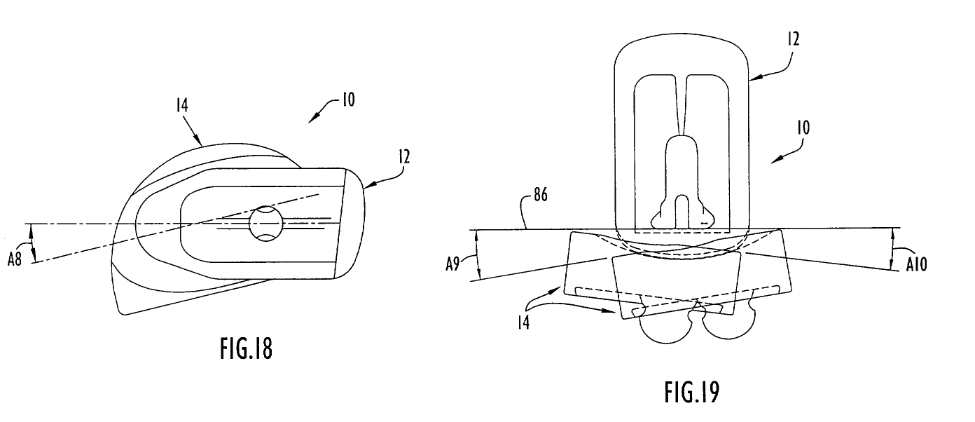

Varus/valgus rotation of the knee joint prosthesis 10 is depicted in FIG. 19, which shows rotation of the femoral component 12 inwardly and outwardly about the medial-lateral center of the tibial articular surface of tibial component 14 until the distal bone surface of the femur, represented by line 86, impinges on the proximal tibial surface. The total range of varus/valgus rotation for the illustrative but not limiting knee joint prosthesis 10 corresponds to the sum of angle A9, which is about eight degrees, and angle A10, which is about seven degrees, so that the range of varus/valgus rotation is about fifteen degrees for the illustrative but not limiting embodiment.

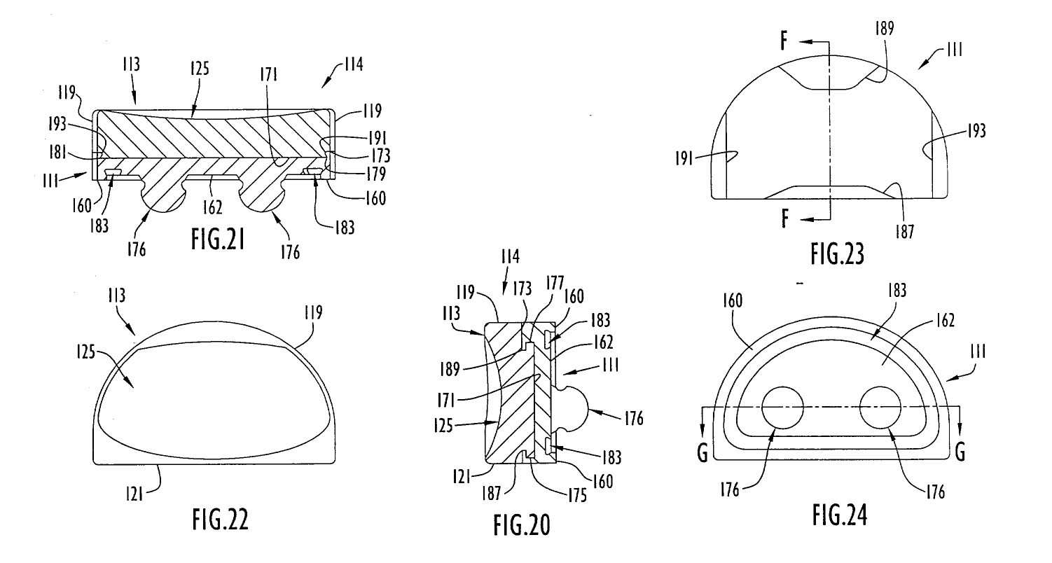

A modified tibial component 114 for use with femoral component 12 in an alternative knee joint prosthesis is shown in FIGS. 20 and 21. Tibial component 114 is a modular tibial component including a tibial base 111 and a tibial insert 113 mounted to base 111. The insert 113 is made in its entirety of a non-metallic, biocompatible weight-bearing material such as polyethylene material, and the base 111 is made of a medically acceptable metal. Insert 113, as shown in FIGS. 20-22, is similar to tibial component 14 and includes tibial articular surface 125, similar to tibial articular surface 25. Insert 113 differs in thickness from tibial component 14 and does not have a tibial fixation surface or peg. Insert 113 has a bottom or distal surface 171 and a lower surface 173 recessed from bottom surface 171 to define inwardly protruding first and second side lip formations 175 and 177, respectively, shown in FIG. 20, and inwardly protruding anterior and posterior lip formations 179 and 181, respectively, as shown in FIG. 21. The first and second side lip formations 175 and 177 are located on the insert at locations corresponding to first and second side shoulder formations of base 111, and the anterior and posterior lip formations 179 and 181 are located on the insert at locations corresponding to anterior and posterior shoulder formations of base 111. For implantation on a medial tibial plateau, the first side lip formation 175 and the first side shoulder formation are lateral formations while the second side lip formation 177 and the second side shoulder formation are medial formations. The lip formation 175 forms a recess extending along a central segment of planar side wall 121, and the lip formation 177 forms an opposing recess extending along a central segment of arcuate side wall 119 as seen in FIG. 20. As shown in FIG. 21, the anterior and posterior lip formations 179 and 181 form opposing recesses extending along anterior and posterior end segments, respectively, of arcuate side wall 119, and the recesses formed by lip formations 179 and 181 extend to the anterior and posterior ends of planar side wall 121. Preferably, the insert is formed integrally, unitarily or monolithically as one piece.

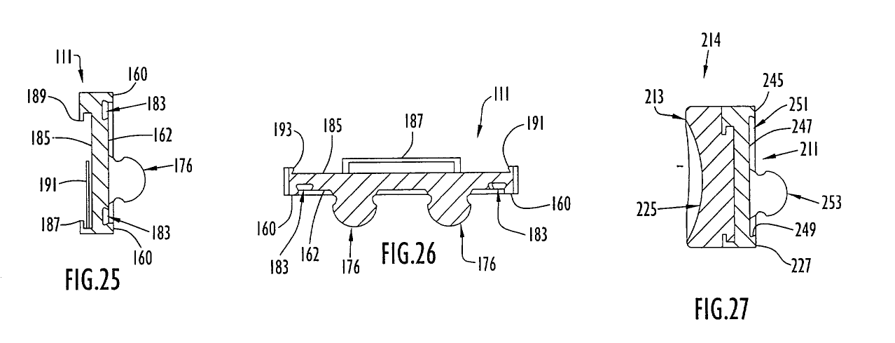

The base 111 has a D-shaped configuration corresponding to the D-shaped configuration of insert 113 and includes a tibial fixation surface having a planar peripheral border 160, a planar surface 162 recessed from peripheral border 160, and a peripheral groove 183 disposed around surface 162 as best shown in FIGS. 20, 21 and 24-26. Tibial fixation pegs 176 extend distally or downwardly from surface 162 and are similar to fixation pegs 53. A pocket or cavity for receiving cement or other bonding or cementitious material is defined between border 160 and surface 162 and by groove 183. Preferably, the base is formed integrally, unitarily or monolithically as one piece.

A top surface 185 of base 111 has upstanding and inwardly protruding first and second side shoulder formations 187 and 189, respectively, best shown in FIGS. 20, 23 and 25. The top surface 185 also has upstanding and inwardly protruding anterior and posterior shoulder formations 191 and 193, respectively, as best seen in FIGS. 21, 23, 25 and 26. The shoulder formation 189 protrudes inwardly from and extends along a central segment of the arcuate side wall of base 111, and the opposing shoulder formation 187 protrudes inwardly from and extends along a central segment of the planar side wall of base 111. The opposing anterior and posterior shoulder formations 191 and 193 protrude inwardly from and extend along the anterior and posterior end segments, respectively, of the arcuate side wall of base 111 and extend to the anterior and posterior ends, respectively, of the planar side wall of base 111. The side shoulder formations 187 and 189 are configured to be received in the recesses defined by the side lip formations 175 and 177, respectively, of insert 113. The side shoulder formations 187 and 189 define notches having a configuration to receive the side lip formations 175 and 177, respectively. The anterior and posterior shoulder formations 191 and 193 are configured to be received in the recesses defined by the anterior and posterior lip formations 179 and 181, respectively, of insert 113. The anterior and posterior shoulder formations 191 and 193 define notches having a configuration to receive the anterior and posterior lip formations 179 and 181, respectively. The insert 113 is assembled onto the base 111 with the lip formations of the insert deflecting to enter the corresponding notches of the shoulder formations and with the bottom or distal surface 171 of the insert upon the top or proximal surface 185 of the base. When thusly assembled, an external periphery of the insert is aligned with an external periphery of the base so that the base does not protrude beyond the external periphery of the insert. The tibial component 114 is shown as an illustrative but not limiting Size 2 tibial component, however, the modular tibial component may be made available in Sizes 1-4, and each size may be made available in different thicknesses, such as 9 mm, 10 mm and 11 mm thicknesses, for example.

Another modified tibial component 214 for use with femoral component 12 in a further alternative knee joint prosthesis is depicted in FIG. 27. Tibial component 214 is a modular tibial component with a tibial articular surface 225 on insert 213 and a tibial fixation surface on base 211 similar to tibial component 114, except that the tibial fixation surface for tibial component 214 is similar to the tibial fixation surface for tibial component 14. Accordingly, the tibial fixation surface 227 for tibial component 214 includes peripheral rim 245, recessed surface 247, and connecting wall 249 forming a dovetail around a cavity 251. In addition, a pair of grooved tibial fixation pegs 253, only one of which is visible in FIG. 27, extend distally, outwardly or downwardly from recessed surface 247.

The articulating interface in the knee joint prostheses of the present invention is a semi-constrained, round-on-round design which improves implant stability and contact area while utilizing bone-conserving femoral and tibial components. The posterior angle and the anterior-posterior angled articular surface of the femoral component enhances anatomical femoral-tibial tracking. The femoral component essentially reproduces the anatomy of the natural femur by providing a constant sagittal radius beyond ten degrees of flexion in combination with the angled anterior-posterior geometry. The geometric couple of the femoral and tibial components provides ligamentous stability throughout a range of motion and increased contact area between the components to promote reduced wear of the tibial articular surface. The anterior-posterior curvature of the tibial articular surface promotes ligamentous stability while allowing sliding or rollback motion, with the amount of sliding or anterior-posterior travel controlled by the distance between the centers of the two offset radii of curvature. Maximum contact area between the femoral and tibial components is maintained while allowing internal-external rotation and/or varus/valgus malalignment between the components. Despite various degrees of component malalignment, maximum contact area is maintained throughout range of motion. The geometric surface of the femoral component mates with a universal tibial geometry such that anatomical femoral components may be used with non-anatomical tibial components. The geometric couple of the tibial and femoral components deters anterior overhang and possible patella articular disruption and also deters medial/lateral overhang. The tangent radii of the femoral articular surface optimize medial kinematics. The geometric configuration of the femoral fixation surface promotes bone conservation by reducing the amount of bone that must be removed from the femoral condyle to accommodate the femoral component. The pockets or cavities of the femoral and tibial fixation surfaces provide an enhanced cement mantle, and the fixation pegs improve fixation to the bone. The fin on the femoral component enhances structural characteristics and integrity while being conservatively designed to minimize bone removal. Proper, reproducible alignment of the femoral component is enhanced via the femoral fixation peg and fin. The dovetail of the tibial component enhances cement fixation. Long term tibial component survivorship and fixation are promoted utilizing a proximal tibial resection and cortical tibial rim support. The tibial component provides high disassociative forces and optimal attributes for cemented fixation. The knee joint prostheses facilitate consistent alignment and reproducible clinical results obtained through an instrumented minimal incision technique.

Inasmuch as the present invention is subject to various modifications and additions, the preferred embodiments are intended to be illustrative only and not limiting since various modifications, variations and changes can be made thereto without departing from the scope of the invention as defined by the appended claims.

What is claimed is:

1. A knee joint prosthesis for implantation in a compartment of a knee comprising
a femoral component for implantation on a prepared femoral condyle of the knee, said femoral component comprising a femoral articular surface having a medial-lateral radius of curvature; and
a tibial component for implantation on a corresponding prepared tibial plateau of the knee, said tibial component comprising a tibial articular surface cooperable with said femoral articular surface to permit motion at the knee between extension and flexion, said tibial articular surface having a medial-lateral radius of curvature 0.25 to 5.0 mm longer than said medial-lateral radius of curvature of said femoral articular surface.

2. The knee joint prosthesis recited in claim 1 wherein said medial-lateral radius of curvature of said tibial articular surface is about 1.0 mm longer than said medial-lateral radius of curvature of said femoral articular surface.

3. A knee joint prosthesis for implantation in a compartment of a knee comprising
a tibial component for implantation on a prepared tibial plateau of the knee, said tibial component comprising a tibial articular surface having a medial-lateral mid-point; and
a femoral component for implantation on a corresponding prepared femoral condyle of the knee, said femoral component comprising a femoral articular surface cooperable with said tibial articular surface to permit motion at the knee between extension and flexion, said femoral articular surface having a tracking line along which said femoral component tracks in an anterior-posterior direction along said medial-lateral mid-point of said tibial articular surface during motion at the knee between extension and flexion, said tracking line tracking at a medial-lateral angle relative to said medial-lateral mid-point of said tibial articular surface.

4. The knee joint prosthesis recited in claim 3 wherein said femoral articular surface has a medial-lateral width and said tracking line is centered on said medial-lateral width of said femoral articular surface.

5. The knee joint prosthesis recited in claim 3 wherein said tracking line tracks at a medial-lateral angle of three to fifteen degrees.

6. The knee joint prosthesis recited in claim 3 wherein said tibial component is adapted for implantation on a prepared medial tibial plateau, said femoral component is adapted for implantation on a prepared corresponding medial femoral condyle, and said tracking line tracks at a lateral angle.

7. The knee joint prosthesis recited in claim 3 wherein said tibial articular surface has a medial-lateral curvature and an anterior-posterior curvature, and said femoral articular surface has a medial-lateral curvature and an anterior-posterior curvature forming a round-on-round tibiofemoral interface.

8. The knee joint prosthesis recited in claim 7 wherein said medial-lateral curvature of said tibial articular surface is constant throughout said tibial articular surface and said medial-lateral curvature of said femoral articular surface is constant throughout said femoral articular surface.

9. The knee joint prosthesis recited in claim 3 wherein said femoral articular surface has an anterior-posterior curvature defined by tangent radii of curvatures.

10. A prosthetic femoral component for implantation in a compartment of a knee comprising
a prosthetic body for implantation on a prepared femoral condyle of the knee, said body comprising a femoral fixation surface for fixation on the prepared femoral condyle, a femoral articular surface cooperable with a tibial articular surface along the corresponding tibial plateau to permit motion at the knee, a posterior portion bisected by a first sagittal plane and a distal portion bisected by a second sagittal plane, said first plane being disposed at an angle to said second plane, said femoral fixation surface comprising a planar posterior section along said posterior portion and a curved section along said distal portion extending anteriorly from said planar posterior section.

11. The femoral component recited in claim 10 wherein said angle is in the range of three to fifteen degrees.

12. The femoral component recited in claim 10 wherein said femoral fixation surface comprises a peripheral border for fixation on the prepared femoral condyle and a cavity circumscribed by said border to receive a cementitious material for fixation of said body to the prepared femoral condyle, said border comprising said planar posterior section, a planar anterior section along said distal portion of said femoral component and said curved section extending from said anterior section to said posterior section.

13. The femoral component recited in claim 12 and further comprising a femoral fixation peg extending outwardly from said femoral fixation surface, said femoral fixation peg being bisected by a plane disposed perpendicular to said second plane.

14. The femoral component recited in claim 13 and further comprising a femoral fixation fin extending outwardly from said femoral fixation surface, said femoral fixation fin comprising an anterior femoral fixation fin segment extending anteriorly from said femoral fixation peg and a posterior femoral fixation fin segment extending posteriorly from said femoral fixation peg, said femoral fixation fin being bisected by said second plane.

15. The femoral component recited in claim 14 wherein said femoral fixation surface further comprises a recessed surface recessed from and circumscribed by said border, said cavity is defined between said border and said recessed surface and said femoral fixation peg and said femoral fixation fin extend outwardly from said recessed surface.

16. The femoral component recited in claim 14 wherein said femoral component has a medial-lateral width and said femoral fixation fin is centered within said medial-lateral width.

17. The femoral component recited in claim 10 wherein said femoral component has a medial-lateral width, said femoral articular surface comprises a highest point centered within said medial-lateral width and said highest point is disposed at said angle to said first plane.

18. A prosthetic femoral component for implantation in a compartment of a knee comprising
a prosthetic body for implantation on a prepared femoral condyle of the knee, said body comprising a femoral articular surface cooperable with a tibial articular surface along the corresponding tibial plateau to permit motion at the knee and a femoral fixation surface for fixation on the prepared femoral condyle, said femoral fixation surface comprising a planar posterior section, an intermediate section extending anteriorly from said posterior section, and an anterior section extending anteriorly from said intermediate section, said intermediate section and said anterior section having a central longitudinal axis disposed in a plane, said planar posterior section being non-perpendicular to said central longitudinal axis.

19. The femoral component recited in claim 18 wherein said planar posterior section is bisected by a bisecting plane disposed perpendicular to said planar posterior section and said central longitudinal axis is angularly offset in a medial-lateral direction from said bisecting plane.

20. The femoral component recited in claim 19 wherein said central longitudinal axis is angularly offset three to fifteen degrees.

21. The femoral component recited in claim 18 wherein said intermediate section is curved and said anterior section is planar.

22. A prosthetic femoral component for implantation in a compartment of the knee comprising
a prosthetic body for implantation on a prepared femoral condyle of the knee, said body comprising a femoral articular surface cooperable with a tibial articular surface along the corresponding tibial plateau to permit motion at the knee between extension and flexion, said femoral articular surface having an anterior-posterior curvature with a constant radius of curvature in a sagittal plane from ten degrees to a range of ninety to one hundred five degrees of flexion at the knee.

23. The femoral component recited in claim 22 wherein said femoral component includes a posterior portion bisected by a first sagittal plane and a distal portion bisected by a second sagittal plane disposed at an angle of three to fifteen degrees with said first sagittal plane.

24. A prosthetic tibial component for implantation in a compartment of a knee comprising
a prosthetic body for implantation on a prepared tibial plateau of the knee, said tibial component comprising a tibial articular surface cooperable with a femoral articular surface along the corresponding femoral condyle to permit motion at the knee, said tibial articular surface having an inward anterior-posterior sagittal curvature comprising a central curved segment disposed between and connected with anterior and posterior curved segments, respectively, said central curved segment having a first radius of curvature and said anterior and posterior curved segments each having a second radius of curvature smaller than said first radius of curvature, said first radius of curvature having a center, said second radius of curvature for said anterior segment having a center offset an offset distance anteriorly from said center of said first radius of curvature, and said second radius of curvature for said posterior segment having a center offset said offset distance posteriorly from said center of said first radius of curvature.

25. The tibial component recited in claim 24 wherein said tibial articular surface is symmetrical about a coronal plane and about a plane perpendicular to said coronal plane.

26. The tibial component recited in claim 24 wherein said tibial articular surface is made of a non-metallic, biocompatible weight-bearing material.

27. The tibial component recited in claim 26 wherein said tibial component is made in its entirety of said non-metallic, biocompatible weight-bearing material.

28. The tibial component recited in claim 26 wherein said tibial component comprises a tibial fixation surface for fixation on the prepared tibial plateau, a base defining said tibial fixation surface and an insert received in said base and defining said tibial articular surface, said base being made of metal and said insert being made of said nonmetallic, biocompatible weight-bearing material.

29. The tibial component recited in claim 24 wherein said tibial articular surface has a medial-lateral curvature and said medial-lateral curvature is constant along said tibial articular surface.

30. A prosthetic tibial component for implantation in a compartment of a knee comprising
a prosthetic body for implantation on a prepared tibial plateau of the knee, said body comprising a tibial articular surface cooperable with a femoral articular surface along the corresponding femoral condyle to permit motion at the knee and a tibial fixation surface for fixation on a planar surface of the prepared tibial plateau, said tibial fixation surface comprising a continuously planar peripheral rim for being continuously supported on the planar surface of the prepared tibial plateau, a recessed surface recessed from and circumscribed by said rim, and a connecting wall connecting said rim to said recessed surface, said connecting wall being angled inwardly from said recessed surface to form a dovetail, said tibial component comprising a cavity defined between said rim and said recessed surface for receiving cementitious material, said cavity being circumscribed by said dovetail, said tibial component comprising a pair of tibial fixation pegs extending outwardly from said recessed surface for placement in the tibia when said rim is supported on the planar surface of the prepared tibial plateau.

31. The tibial component recited in claim 30 wherein said recessed surface is planar.

32. The tibial component recited in claim 30 wherein said connecting wall is angled about 45 degrees relative to said recessed surface.

33. The tibial component recited in claim 30 wherein said tibial fixation pegs are formed integrally, unitarily with said tibial component and said tibial component is made in its entirety of a non-metallic, biocompatible weight-bearing material.

34. The tibial component recited in claim 30 wherein said tibial component comprises an insert made of non-metallic, biocompatible weight-bearing material and defining said tibial articular surface and a base receiving said insert, said base defining said tibial fixation surface and said tibial fixation pegs and being made of metal, said tibial fixation pegs being formed integrally, unitarily with said base.

35. The tibial component recited in claim 30 wherein said tibial component has a D-shaped peripheral configuration with a curved side wall and a straight side wall.

36. The tibial component recited in claim 30 wherein said tibial fixation pegs comprise a posterior tibial fixation peg and an anterior tibial fixation peg.
